(19) United States
(12) Patent Application Publication
(10) Pub. No.: US 2004/0186436 A1
(43) Pub. Date: Sep. 23, 2004

(54) Devices for administering material

(76) Inventor: John Leyshon Maddocks, Endcliffe (GB)

Correspondence Address: SCHWARTZ LAW FIRM, P.C., 6100 FAIRVIEW ROAD, SUITE 530, CHARLOTTE, NC, 28210, US

(21) Appl. No.: 10/482,471

(22) PCT Filed: Jun. 18, 2002
(86) PCT No.: PCT/GB02/02776

(30) Foreign Application Priority Data
Jun. 30, 2001 (GB) 0116036.5

(51) Int. Cl. A61M 5/00

(52) U.S. Cl. 604/187

(57) ABSTRACT

A device (10) is provided for use in controlling the administration of material by administration apparatus. The device is selectively operable between a first condition in which the administration of material from the apparatus (10) is prevented and a second condition which allows material to be dispensed from the apparatus (10). The device also comprises means (13) indicating appropriate administration from the apparatus (12).

The present invention relates to devices for administering material and in particular but not exclusively to devices for use in the administration of material such as medicine or other therapeutic material to a body, such as the human or animal body.

The administration of medicine or other therapeutic material, such as blood, to the human or animal body requires care, particularly to ensure that the material is appropriately administered to the body.

Sometimes inappropriate administration of material can lead to serious difficulties and complications to the body to which it is administered. For example, if the route of administration of the material is inappropriate this can cause difficulties, and indeed can be fatal. For example, the cytotoxic drug Vincristine requires to be injected intravenously, but there have been instances when this drug has been accidentally injected into the spine in error with often fatal consequences. Other examples include the administration of local anaesthetic Bupivacaine which when injected intravenously instead of in the epidura can be fatal. Similar injection errors have occurred with other drugs such as adrenaline, potassium chloride and neuro-muscular blocking agents.

Many approaches have been considered to address these issues, which arise predominantly because drugs are administered by humans and humans are prone to human error. Increases in complexity of procedures and protocols for drug administration has been one approach, but these can sometimes in themselves establish environments in which errors are likely to occur.

The administration of materials to other bodies or systems can often also require extreme care to ensure appropriate administration. For example, the addition of chemicals to water systems, constituents to foodstuffs and drinks etc. can sometimes require very careful administration to prevent potentially hazardous or otherwise undesirable situations arising.

According to the present invention there is provided a device for use in controlling the administration of material by administration apparatus, the device being selectively operable between a first condition in which the administration of material from the apparatus is prevented and a second condition which allows material to be dispensed from the apparatus and comprising means indicating appropriate administration of material from the apparatus.

Preferably the device is located for use upon administration apparatus. Preferably the device is selectively moveable between the first and second conditions.

Preferably the device is moveable upon appropriate manipulation of the device.

The device may comprise lock means operable to selectively releasably secure the device in the first condition.

The device may further comprise input means with which a user can manipulate the device to enable selective securing and release of the lock means. The input means may comprise mechanical means, such as manually operable members, for example rotatable lock tumblers. Alternatively or in addition the lock means may be electronically operable.

The device may comprise comparison means to compare information inputted into the device with predetermined information accessible to the comparison means and to enable the device to be moved to the second condition when the inputted information is deemed appropriate by the comparison means. The comparison means may comprise a memory to hold information. The memory may hold predetermined information, such as information relating to the material to be administered, the body, such as a patient, to which the material is to be administered. Alternatively or in addition some or all of the predetermined information may be held remotely from the device to be accessible thereby.

Preferably the indicating means comprises a sign such as one or more letter(s) and/or symbol(s) advising an observer of the correct administration of the material using the apparatus. Preferably the indicating means notifies an observer, in particular a user of the device, of the correct route of administration of material using the apparatus.

Preferably the indicating means advises, either directly or indirectly, the user of the device of the appropriate manipulation of the device required to unlock the lock means.

The indicating means may comprise a string of symbols relating to the administration of material with the apparatus, which string of symbols when appropriately aligned on the input means enable, release of the lock means.

The input means may comprise means to send electronic signals to unlock the lock means, and may comprise buttons, keys or similar means. The device may comprise an electronic display, which may provide the indicating means and/or display data inputted into the device.

The device may comprise transmitter means to send signals to remote data processing apparatus, such as a computer, server, Internet apparatus or the like. The device may comprise receiver means operator to receive signals, for example signals sent from a computer, server or such means, which signals may be processed by processing means provided in the device.

The device may comprise a body locatable, in use, around a section or part of administration apparatus. The device is preferably locatable in use on the plunger of a syringe used to administer material. Alternatively or in addition the device may be locatable in use on a material supply line, such as an ivi line.

The body preferably comprises a first part movably attached to a second part with the lock means operable to secure the parts around the aspects in the first condition. The parts may be pivotally attached, with the lock means preferably acting to clamp the parts around the apparatus when in the first condition.

Preferably the device prevents or restricts movement of the plunger into the syringe when in the first condition on the syringe whereby to prevent administration of the material in the syringe.

The device may further comprise a remote control unit, such as a computer, palm top computer, telephone, mobile telephone, pager or similar such device to enable remote control of the body.

The invention also provides apparatus for administering material to a body, the apparatus comprising a device as described in any of the preceding paragraphs.

Preferred embodiments of the present invention will now be described by way of example only, with reference to the accompanying drawings in which:-

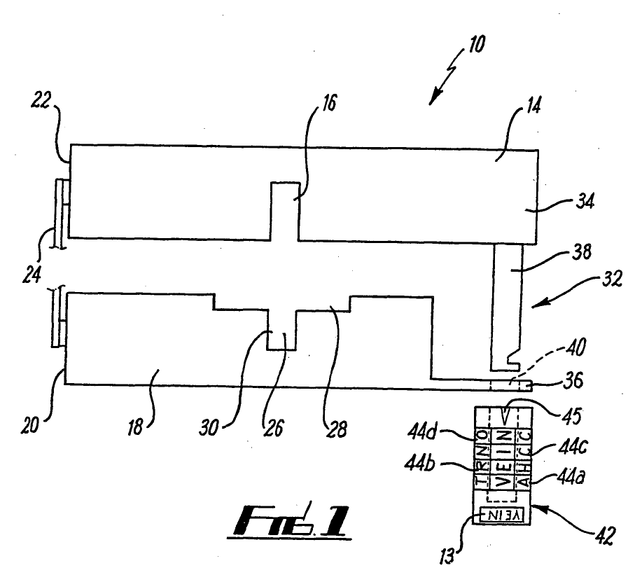

FIG. 1 is a diagrammatic exploded view of a device according to the present invention;

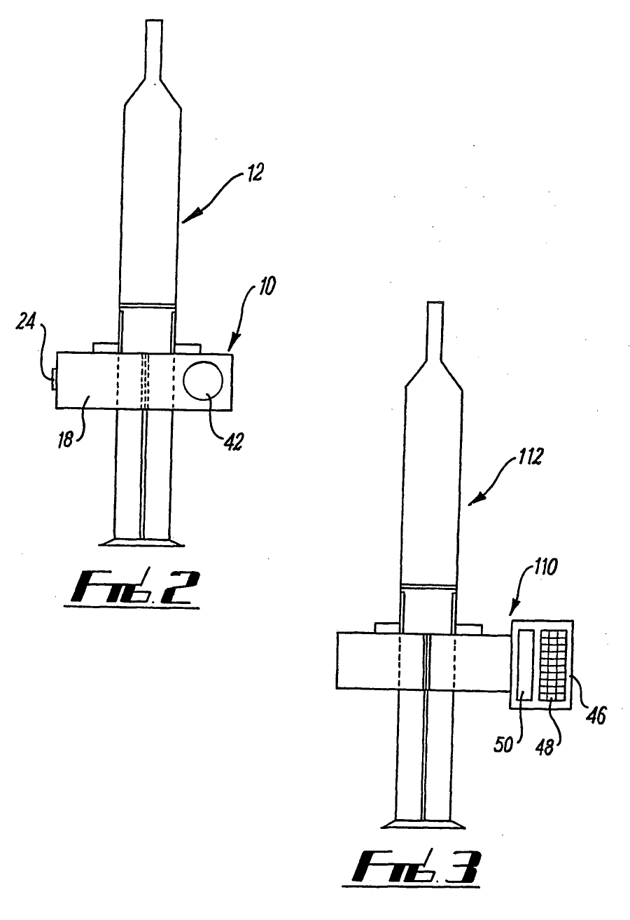

FIG. 2 is a reduced scale diagrammatic representation of the device of FIG. 1 in use on a syringe; and

FIG. 3 is a view similar to FIG. 2 showing a device according to an alternative embodiment of the present invention.

Referring to the drawings, there is provided a device 10 for use in the administration of material to a body (not shown), the device 10 being located for use upon administration apparatus, shown in the form of a syringe 12 (FIG. 2) to be selectively moveable between a first condition in which the administration of material from the apparatus 12 is prevented and a second condition which allows material to be dispensed from the apparatus 12 and comprising means 13 indicating appropriate administration of material from the apparatus 12.

In more detail, the device 10 comprises a first part 14 of generally rectangular cross-section and comprising a recess 16 on one side generally mid-way along its length.

A second part 18 is pivotally attached at one end 20 to an end 22 of the part 14 by a linkage 24 (which is shown broken in FIG. 1 but which would provide pivotal connection between the two parts 14 and 18).

A re-entrant portion 26 is provided generally mid-way along the length of a side of the second part 18. The re-entrant portion 26 comprises a shallow portion 28 generally from the centre of which extends a narrower portion 30 into the body of the part 18. Between the recess 16 and re-entrant portion 26 is defined a generally cruciform section to receive and therefore allow the device 10 to locate around, the plunger of syringes which conventionally have cruciform cross-sections. It will however be appreciated that the shape of the section defined between the parts 14, 18 can be varied and determined according to the shape of the apparatus or part thereof around which the device 10 is to locate in use.

Lock means 32 is provided to be operable between the respective other ends 34, 36 of the part 14, 18. The lock means 32 comprises a shaft 38 extending from the end 34 to be locatable through an opening 40 in the end 36 of the other part 18 to receive a tumbler mechanism 42 of generally known design. The lock means 32 itself is of generally known design. The tumbler mechanism 42 comprises a series of four finger moveable tumblers 44 a, b, c, d around each of which is provided a series of letters of the alphabet.

The indicating means 13 comprises a sticker bearing a word, in this example ‘vein’ indicating the appropriate route for administration of material.

In use, the device can be used in hospitals and other institutions where drugs and other therapeutic material are administered to humans, or indeed animals.

Particularly with regard to drugs, these are often dispensed by pharmacists or similar practitioners who load the appropriate material and dosage into the apparatus that will be used to administer it, in most cases a syringe. This stage is usually double checked by a second pharmacist.

Once the syringe has been loaded with material, the pharmacist applies the sticker 13 on the device 10, preferably in the vicinity of the tumbling mechanism 42. In this particular example the material is to be administered intravenously, and therefore an appropriate indication to the person who is to, administer the material (which is not the pharmacist) is ‘vein’ and the sticker bears this word.

It will of course be appreciated that any appropriate indication, sign or symbol could be used to indicate appropriate administration although it is preferable that the meaning of any indication used renders it obvious to personnel who will be administering the material what the correct administration is. Another example could be the code ‘thec’ to indication intrathecal administration.

The device 10 is then securely located around the plunger of the syringe 12 with the plunger 12 located within the opening defined between the recess 16 and re-entrant portion 26, and the lock means 32 used to secure the device 10 in this first position. The precise location of the device 10 on the plunger 12 is determined according to the degree of movement of the plunger in the cylinder of the syringe that is to be prevented.

The syringe 12 and the material therein are now secure against inappropriate administration. The device 10 can only be removed from the first condition around the syringe 12 when the lock means 32 is released, which in this example require the tumblers 44 a , b, c, d to be appropriately manipulated to align the letters of the four tumbler 14 a, b, c and d to spell out, at the appropriate location, the word ‘vein’. A mark 45 adjacent the tumbler indicates the location of alignment.

It will be seen that in use, personnel to administer material from apparatus 12 secured with the device 10 have to consciously insert the correct route of administration to unlock the device 10 from the first condition and thereby allow the device 10 to be moved from around the plunger and thus allow administration of the material from the syringe. The requirement for this step ensures that personnel administering the material consciously note the route of administration before administering the material which significantly reduces the possibility of inappropriate administration.

FIG. 3 shows an alternative embodiment of a device 110, similar to device 10 but which comprises an electronically operable lock means 46 which comprises an array of buttons or keys 48 in which data can be entered to release the lock means 46 and a display 50 which is operable as indicating means to display the appropriate administration, and which may also display other data, such as the entries being made by a user to release the device 110.

The lock means 46 may be of any appropriate design, and the device 110 functions in essentially the same way as the device 10 described above.

Additional features may be incorporated with electronic lock means 46. For example, the device 110 may be able to display and/or receive further data such as patient number, patients history, details of the material being administered, drug contraindications, drug interactions, dosage and the like. However, it is important that the device 110 displays the appropriate administration and requires the personnel to administer material to consciously note and input appropriate data to release the lock means 46 and thereby enable administration.

It will be appreciated that the indicating means could be used to indicate other aspects of administration, other than or in addition to the route of administration. It could for example identify the patient to whom material is to be administered.

It will further be appreciated that particularly with the electronic device 110 data could be transmitted to remote data processing equipment, such as a computer server Internet apparatus and the like, which could record information from and send information to the device, including date and time of administration, administrator, etc. The device can also comprise receiver means to receive and process data signals, for example signals sent by a remote computer server or the like.

The device may also comprise a remote controller, such as a hand held device like a palm top computer, telephone, mobile telephone, pager, computer or similar such device to enable remote control of the body on the administration apparatus.

The device may be operable using infra red and/or radio frequency signals.

In a further embodiment of the invention the device comprises comparison means, such as a computer processor unit, which is operable to compare information inputted into the device by a user with predetermined information accessible to the comparison means. The comparison means controls movement of the device to the second condition, only allowing the device to assume the second condition when information inputted into the device is appropriate and compares favourably with the predetermined information. The predetermined information can be stored in a memory of the device and/or remotely, for example in (a) computer database(s) or the like. The predetermined information can relate to any aspect that is relevant to the appropriate administration of material from the apparatus, such as information relating to the body to which material is to be administered including patient information, drug therapy, allergies, information relating to the material to be administered.

In use, the device of the embodiment could be regarded by a user as effectively the body to which material is to be administered. In this way, a user inputs information into the device as if it were administering material. If the inputted information is considered correct and appropriate by the comparison means, the means moves or enables the device to be moved to the second condition where the apparatus on which the device is operable can administer material therefrom.

It is also within the scope of the present invention that a plurality of devices could be used on a single administration apparatus providing still further security against inappropriate administration.

The invention can also be used in the administration of material to any other body, system, arrangement or composition, particularly where material has to be carefully administered. For example the invention could be used in the administration of one or more chemicals into fluid systems such as water systems and water treatment systems, the administration of ingredients into beverages and foodstuffs.

Whilst endeavouring in the foregoing specification to draw attention to those features of the invention believed to be of particular importance it should be understood that the Applicant claims protection in respect of any patentable feature or combination of features hereinbefore referred to and/or shown in the drawings whether or not particular emphasis has been placed thereon.

1. A device for use In controlling the administration of material by administration apparatus, the device comprising a body locatable around a section or part of administration apparatus, said body being selectively movable between a first condition in which the administration of material from the apparatus is prevented and a second condition which allows material to be dispensed from the apparatus, and the apparatus further including means indicating appropriate administration of material from the apparatus, and lock means operable to selectively releasably secure the body in the first condition, wherein the lock means is configured to be operable to release the body and allow the body to move to the second condition only on inputting to the lock means a combination of symbols relating to the administration of the material.

2. A device according to claim 1, wherein the combination of symbols and the indicating means correspond to one another.

3. A device according to claim 1 or 2, wherein the lock means comprises Input means for inputting to the lock means said combination of symbols.

4. A device according to claim 3, wherein the input means comprises a plurality of input members for inputting the combination of symbols to the lock means.

5. A device according to claim 3 or 4, in which the input means comprises mechanical means.

6. A device according to claim 5, in which the input means composes rotatable lock tumblers.

7. A device according to claim 3 or 4, in which the lock means may be electronically operable.

8. A device according to claim 7, in which the device comprises comparison means to compare information inputted into the device with predetermined information accessible to the comparison means and to enable the device to be moved to the second condition when the inputted information is deemed appropriate by the comparison means.

9. A device according to claim 8, in which the comparison means comprises a memory to hold information.

10. A device according to claim 9, in which the memory may hold predetermined information.

11. A device according to claim 10, in which the memory holds information relating to the material to be administered, the body, such as a patient, to which the material is to be administered.

12. A device according to any of claims 8 to 11, in which some or all of the predetermined information is held remotely from the device to be accessible thereby.

13. A device according to any of claims 2 to 12, wherein the indicating means comprises a sign such as one or more letter(s) and/or symbol(s) advising an observer of the correct administration of the material using the apparatus.

14. A device according to claim 13, in which the indicating means notifies an observer, in particular a user of the device, of the correct route of administration of material using the apparatus.

15. A device according to claim 13 or 14, in which the indicating means advises, either directly or indirectly, the user of the device of the appropriate manipulation of the device required to unlock the lock means.

16. A device according to claim 15, in which the indicating moans comprises a string of symbols relating to the administration of material with the apparatus, which sting of symbols when appropriately aligned on the input means enable release of the lock means.

17. A device according to any of claims 7 to 16, in which the input means comprises means to send electronic signals to unlock the lock means.

18. A device according to any claims 3 to 17, in which the input means comprises buttons, keys or similar means.

19. A device according to any preceding claim, in which the device comprises an electronic display.

20. A device according to claim 19, in which the display provides the indicating means and/or display data inputted into the device.

21. A device according to any of claims 7 to 20, in which the device comprises transmitter means to send signals to remote data processing apparatus, such as a computer, server, internet apparatus or the like.

22. A device according to any of claims 7 to 21, in which the device comprises receiver means operator to receive signals, for example signals sent from a computer, server or such means.

23. A device according to claim 22, in which the device comprises processing means to process received signals.

24. A device according to any preceding claim, in which the device is locatable in use on the plunger of a syringe used to administer material.

25. A device according to any of claims 1 to 23, in which the device is locatable in use on a material supply line, such as an ivi line.

26. A device according to any preceding claim, in which the body comprises a first part movably attached to a second part with the lock means operable to secure the parts around the apparatus in the first condition.

27. A device according to claim 26, in which the parts are pivotally attached.

28. A device according to claim 26 or 27, in which the lock means acts to clamp the parts around the apparatus when In the first condition.

29. A device according to claim 24, in which the device prevents or restricts movement of the plunger into the syringe when in the first condition on the syringe whereby to prevent administration of the material in the syringe.

30. A device according to any of claims 7 to 29, in which the device comprises a remote control unit such as a computer, palm top computer, telephone, mobile telephone, pager or similar such device to enable remote control of the body.

31. An assembly for administering material to a body, comprising administration apparatus and a device as claimed in any of the preceding claims.

32. A device substantially as hereinbefore described with reference to the accompanying drawings.

33. Apparatus substantially as hereinbefore described with reference to the accompanying drawings.
